(19) United States
(12) Patent Application Publication
(10) Pub. No.: US 2017/0020178 A1
(43) Pub. Date: Jan. 26, 2017

(54) Nutritionally and Botanically Enhanced Microbial/Bacterial Biomass

(71) Applicant: Functional Fungi, LLC, Arroyo Grande, CA (US)

(72) Inventor: Jordan Seth Rubin, Koshkonong, MO (US)

(21) Appl. No.: 15/076,931

(22) Filed: Mar. 22, 2016

Related U.S. Application Data
(60) Provisional application No. 62/138,099, filed on Mar. 25, 2015.

(51) Int. Cl. A23L 29/00; A61K 35/742; A23P 10/30; A61K 9/00; A23L 33/135; C12N 1/20; A61K 35/747

(52) U.S. Cl. CPC A23L 29/065; C12N 1/20; A61K 35/742; A61K 35/747; A61K 9/0056; A23L 33/135; A23P 10/30; A23V 2002/00; A23Y 2220/67

(57) ABSTRACT

The present invention is an improved probiotic nutritional supplement, in unit dosage form for animal or human consumption, and a method of preparing it. Any of the nutritionally appropriate bacteria, such as but not limited to Bacillus subtilis, are cultured in connection with novel, enhanced-nutrition media and, in many cases, the enhanced-nutrition media is retained in the end product after fermentation. The enhanced-nutritional media, for example, may contain one or more of a legume, aloe vera juice or other fruit or vegetable juice, turmeric, green tea—or one of many other media constituents that probiotic bacteria have not been cultured on or with previously, for the purpose of preparing unit dosage forms thereof.

### BACKGROUND OF THE INVENTION

Field of the Invention

The invention relates to improved cultivation for edible probiotic fermented foods including microbial/bacterial biomass.

Description of Related Art

Everything old is new again—or are those old things surprisingly better now? Traditional centuries-old probiotics (before they were even called probiotics) included milk products initially fermented by serendipity on long, hot journeys (yogurt, cheese), unfiltered and unrefined fermentation products such as meade and other beverages that still contained their (mostly) beneficial live cultures, and a stunning array of healthful nutrient-containing fermented foods from Asia including but by no means limited to kim chee from Korea, miso (fermented soy) from Japan, fermented shrimp paste from Thailand, nuoc mam fermented fish sauce from Viet Nam, and on and on. The ubiquity of these fermented products speaks volumes to their importance in the healthy diets of literally billions of people for hundreds if not thousands of years.

Notwithstanding the foregoing, a typical limitation in prior art fermentation processes is a constrained nutrient profile. Often, fermentation products known in the prior art contain a very similar nutritional profile as their starting constituents (the basic ingredients plus the biological “starter,” whatever it is) together with some desirable fermentation products and constituent derivatives. By “constituent derivative” is meant here a substance, composition or molecule which has been changed, from the initial food ingredient, by the action of bacteria or other microbe. One of a countless number of constituent derivatives is, by way of non-limiting example, the cheese-making acidification product lactic add, present in most cheese types, instead of the lactose present in the starting milk or cream constituents. It is no wonder so many civilizations, made up of otherwise fresh-milk-avoiding adults, emphasize cheese and yogurt as a dairy staple. Even so, “nutrients in equals nutrients out” has been the basic rule of fermentation both historically, in traditional food preparation, and also in the burgeoning nutritional supplement business which is thriving at this writing.

Stated a different way, traditional growth media for edible probiotic fermented foods has conventionally included a variety of substrates, including but not limited to soy, dairy, and various sugars, but until now the engineering of fermented foods as to a specialized constituency has been in its infancy. It simply has not been known, prior to this writing, what can be added to bacterial growth substrate that can be taken up in the bacterial biomass and preserved into the fermented food—not just added to the end product as an exogenous “additive.” At this writing probiotics and fermented foods are enjoying great popularity, due to their character as a robust natural substance which can be organically grown, safely maintained and shipped, and dried without loss of essential bioactive ingredients. Because probiotic fermented foods can be exceptional nutritional and pharmaceuticals of long shelf life and notable quality, a need remains for ways of growing bacterial biomass having desired composition as well as unusually hearty and fast growth characteristics.

An important need persists, therefore, for a way to enhance the nutritional profile of fermented food substances, and their underlying “starters” (bacteria, yeast, fungi and any other applicable microbes) by growing such organisms and their fermentation products in such a way that their nutritional profiles are naturally enhanced (that is, are not simply supplemented with extraneous, non-naturally-occurring substances or molecules) in fully natural (and ideally certified organic) growth procedures that provide new and surprisingly improved nutritional results.

### SUMMARY OF THE INVENTION

In order to meet this need, the present invention is an innovative method of growing certain strains of bacteria on newly specified media to create heretofore unattainable robust biomass having desired nutritional and pharmaceutical (nutraceutical) profiles. The bacteria grown according to the invention are selected from the group consisting of: Bacillus subtilis, Bacillus subtilis ssp Natto, Bacillus coagulans, and Lactobacillus plantarum. Further according to the invention, these bacterial species are not fermented just in traditional dairy (yogurt, cheese) fruit juice (wine) or simple grain (beer), but are deliberately grown on novel, enhanced-nutrition media selected from the group consisting of (or preponderantly containing one or more of) Kidney Bean; Pumpkin Seed; Fava Bean; Chia Seed; Green Split Pea; Flax Seed; Green Lentil; Garbanzo Bean; Hemp Seed; Black Bean; Mung Bean; Adzuki Bean; Sesame Seed; Cranberry Bean; Great Northern Bean; Lima Bean; Navy Bean; Pinto Bean; Black rice; Ashwaghandha; Cacao nibs; Purple Millet, Black Quinoa, Red Quinoa, Red Canihua, Cannabis Sativa/Indica flowers or leaves; Spirulina; Lemon Peel; Chili Pepper; Kelp; Alfalfa leaf; Watercress; Cilantro; Sage; Thyme; Parsley; Broccoli Seed; Mate; green Coffee Beans; Roasted Coffee Beans; Green Tea; Rhodiola; Siberian Ginseng (Eluetherococcus senticocus); Sea Buckthorn Berry; Black Soy Bean; Olive leaf; Cabbage; Milk Thistle seed; Milk Thistle leaf; Turmeric; Bupleurum; Artichoke leaf; Dandelion leaf; Dandelion root; Hibiscus flower; Ginkgo leaf; Bacopa; Periwinkle; Peppermint; Hawthorn Berry; Cardamon; Garlic; Mung Bean; Astragalus Root; Echinacea purpurea root; Echinacea augustifolia root; Echinacea augustifolia leaf; Elder berries; Elder Flower; Goldenseal Leaf; Goldenseal root; Olive Leaf; Ginger; Orange Peel and Blossom; Clove; Cinnamon; Fennel; Kelp; Bitter Orange/Bergamot; Hibiscus Flower; Frankincense tears (Boswellia); Holy Basil; Celery seed; Bilberry leaf; Gymnema sylvestre leaf; Fennugreek; Bitter Melon; Saw Palmetto; Nettle: Red Clover Blossom; Black Cohosh; Chaste Tree Berries; Dong Quai Root; Hop Flowers; Licorice root; Wild Yam; Thyme Leaf; Oregano; Hyssop; Marjoram; American Ginseng; Panax Ginseng—red and white; Cruciferous vegetables; Schizandra; Goji Berry; Tribulus; Velvet Bean; Cranberry; Almonds; and Epimidium. In a preferred embodiment of the invention, the one or more of Bacillus subtilis, Bacillus subtilis ssp Natto, Bacillus coagulans, and Lactobacillus plantarum are grown not only on one or more of the above nutritionally advanced media but are fermented with both the media and at least one organic juice selected from the group consisting of Organic Carrot Juice and Concentrate; Organic Concord Grape Juice; Organic Pomegranate juice and Concentrate; Organic Ginger Juice; Organic Blueberry Juice and/or Puree; Organic Acai Puree; Organic Plum Puree; Organic Raspberry Puree; Organic Tomato Juice and/or Puree; Organic Cranberry Juice; Organic Coconut Cream; Organic Coconut Water; Organic Cherry Juice; Organic Green Tea; Organic Apple Juice; Organic Vegetable Juice Blend; Organic Fruit juice Blend; Organic Pineapple Juice; and Organic Aloe Vera Juice. The growth and stability of the listed bacteria on the listed medium gives new and unexpected results not only in bacterial constituency but in strength and viability of bacterial biomass and faster and more robust growth than the same bacteria can achieve on traditional, generally single-media growth substrates. There are nutritional compositions and compounds in the resulting fermentation product that simply could not be there, especially in the preferred embodiment employing both growth media and juice and co-fermentation of both, were it not for the fermentation's having generated such a beneficial nutritional compound or composition during processing.

### DETAILED DESCRIPTION OF THE INVENTION

The present invention comprises growing certain probiotic bacteria and edible fermented foods on specialized substrates (media) in the nature of—rather than traditional simple dairy, grain or vegetable substrates—HERBS, SPICES, FRUITS, VEGETABLES, ALGAE, LEGUMES, GRAINS AND SEEDS, ideally fermented in conjunction with one or more nutritionally advantageous juices such as Organic Carrot Juice and Concentrate; Organic Concord Grape Juice; Organic Pomegranate Juice and/or Concentrate; Organic Ginger Juice; Organic Blueberry Juice and/or Puree; Organic Acai Puree; Organic Plum Puree; Organic Raspberry Puree; Organic Tomato Juice and/or Puree; Organic Cranberry Juice; Organic Coconut Cream; Organic Coconut Water; Organic Cherry Juice; Organic Green Tea; Organic Apple Juice; Organic Vegetable juice Blend; Organic Fruit Juice Blend; Organic Pineapple Juice; or Organic Aloe Vera Juice. As described above, the present invention is an innovative method of growing certain strains of bacteria on newly specialized media to create heretofore unattainable robust biomass having desired nutritional and pharmaceutical (nutraceutical) composition. The bacteria grown according to the invention are selected from the group consisting of Bacillus subtilis, Bacillus subtilis ssp Natto, Bacillus coagulans, and Lactobacillus plantarum. According to the invention, these bacterial varieties are grown on media selected from the group consisting of (or preponderantly containing one or more of) Kidney Bean; Pumpkin Seed; Fava Bean; Chia Seed; Green Split Pea; Flax Seed; Green Lentil; Garbanzo Bean; Hemp Seed; Black Bean; Mung Bean; Adzuki Bean; Sesame Seed; Cranberry Bean; Great Northern Bean; Lima Bean; Navy Bean; Pinto Bean; Black rice; Ashwaghandha; Cacao nibs; Purple Millet, Black Quinoa, Red Quinoa, Red Canihua, Cannabis Sativa/Indica flowers or leaves; Spirulina; Lemon Peel; Chili Pepper; Kelp; Alfalfa leaf; Watercress; Cilantro; Sage; Thyme; Parsley; Broccoli Seed; Mate; Green Coffee Beans; Roasted Coffee Beans; Green Tea; Rhodiola; Siberian Ginseng; Sea Buckthorn Berry; Black Soy Bean; Olive leaf; Cabbage; Milk Thistle seed; Milk Thistle leaf; Turmeric; Bupleurum; Artichoke leaf; Dandelion leaf; Dandelion root; Hibiscus flower; Ginkgo leaf; Bacopa; Periwinkle; Peppermint; Hawthorn Berry; Cardamon; Garlic; Mung Bean; Astragalus Root; Echinacea purpurea root; Echinacea augustifolia root; Echinacea augustifolia leaf; Elder berries; Elder Flower; Goldenseal Leaf; Goldenseal root; Olive Leaf; Ginger; Orange Peel and Blossom; Clove; Cinnamon; Fennel; Kelp; Bitter Orange/Bergamot; Hibiscus Flower; Frankincense tears; Holy Basil; Celery seed; Bilberry leaf; Gymnema sylvestre leaf; Fennugreek; Bitter Melon; Saw Palmetto; Nettle; Red Clover Blossom; Black Cohosh; Chaste Tree Berries; Dong Quai Root; Hop Flowers; Licorice root; Wild Yam; Thyme Leaf; Oregano; Hyssop; Marjoram; American Ginseng; Panax Ginseng—red and white; Cruciferous vegetables; Schizandra; Goji Berry; Tribulus; Velvet Bean; Cranberry; Almonds; and Epimidium, and growing the bacteria in this way gives new and unexpected results not only in bacterial constituency but in strength and viability of the fermented food/microbial biomass and faster and more robust growth than the same bacterial fermented food can achieve on traditional growth substrates.

Certified organic biologically active nutraceutical raw material compounds are produced by inoculating herbs, spices, fruits, teas, vegetables, algae, grains, seeds and dairy products with a range of cultures of functional bacterial species (MicroBiome Tranformation System, or “MTS”). The living cultures pre-digest the foods and herbs transforming and potentiating these ingredients. Grains and seeds are sprouted/germinated prior to inoculation. The MTS uses organic whole food ingredients, with zero filler ingredients, zero inactive ingredients, no isolated chemicals, no extracts resulting in a full spectrum potentiation of organic food factors (NewTrients). Products created using the MTS can be vegetarian/vegan friendly. Ingredients are not mixed together, they are fused together by a natural live fermentation process. Raw materials derived from this process are truly whole and truly live. The culture is never killed/terminated with enzymes or heat, that is, materials are truly “living”, and are dried at low temperatures between 105-118 degrees F. to maintain live cultures therein. As explained above, these nutrient rich raw materials are preferably grown using organic juices in addition to the base media. In the further preferred embodiments of the invention, during the cultivation/fermentation process, music is played in the vicinity of the fermentation, providing frequency generating vibrations to impart energy into the finished products. The MTS process creates whole foods rich in vitamins, amino acids, peptides, trace minerals, probiotics, enzymes, antioxidant enzymes, polysaccharides, chelated mineral activators may be added which then greatly enhance the bioavailability of beneficial compounds taken up from the specified media.

As described above, in preferred embodiments of the invention the growth media is supplemented with an organic juice constituent during the fermentation process. These organic juices may be selected from the group consisting of Organic Carrot Juice and Concentrate; Organic Concord Grape Juice; Organic Pomegranate Juice and/or Concentrate; Organic Ginger Juice; Organic Blueberry Juice and/or Puree; Organic Acai Puree; Organic Plum Puree; Organic Raspberry Puree; Organic Tomato Juice and/or Puree; Organic Cranberry Juice; Organic Coconut Cream; Organic Coconut Water; Organic Cherry Juice; Organic Green Tea; Organic Apple Juice; Organic Vegetable juice Blend; Organic Fruit Juice Blend; Organic Pineapple Juice; or Organic Aloe Vera Juice.

### DESCRIPTION OF PARTICULAR PRODUCTS CREATED BY THIS INVENTION

Bacillus subtilis grown on a nutrient dense combination of legumes, seeds and herbs and one or more juices: Multiple health enhancing compounds can be fused together by fermenting Bacillus subtilis on a multi-constituent media containing a choice of legumes, seeds and herbs and one or more juices from the above media list. When Bacillus subtilis is grown this way, rapid proliferation of bacterial biofilm occurs during the cultivation/fermentation process. When Bacillus subtilis is grown on a mixture of organic legumes, seeds, herbs and juices, growth is surprisingly fast and the biomass becomes robust at a speed that is unusual and unexpected.

“Real Probiotic” blend is an encapsulated formula containing Kidney Bean; Garbanzo Bean; Black Bean; Sunflower Seed; Turmeric; Green Tea; Astralagas in a base of the juice from Aloe Vera Inner Leaf, fermented with one of the bacteria disclosed above. Rapid and dense growth of bacterial biofilm occurs during the cultivation/fermentation process.

“Real Superfood” contains Kidney Bean; Pumpkin Seed; Fava Bean; Chia Seed; Green Split Pea; Fax Seed; Green Lentil; Garbanzo Bean; Hemp Seed; Black Bean; Mung Bean; Adzuki Bean; Sesame Seed; Cranberry Bean; Great Northern Bean; Lima Bean; Navy Bean; Pinto Bean; Turmeric, Green Tea and Chili Pepper in a base of organic juices (Grape, Apple, Blueberry, Pomegranate, Blackberry, Cranberry, Lemon, Acerola Cherry, Celery, Purple Carrot, Green Leaf Lettuce, Red Beet, Purple Cabbage, Kale, Collards, Spinach, Watercress, Parsley) and Rose Hips. This complex combination is inoculated with Bacillus subtilis and rapid and dense growth of bacterial biofilm occurs during the cultivation/fermentation process.

Bacillus subtilis Solid Substrate Delivery System: 25-97% of total biomass on a dry weight basis: contains a single or combination of, legumes, seeds, herbs and spices. An exemplary combination is at least five or more of Cabbage, Carrot, Beet, Celery, Onion, Red Canihua, Red Quinoa, Black Quinoa, Black/Purple Rice, Purple Millet, Kidney Bean, Pumpkin Seed, Fava Bean, Chia Seed, Green Spot Pea, Fax Seed, Green Lentil, Garbanzo Bean, Hemp Seed, Black Bean, Mung Bean, Adzuki Bean, Sesame Seed, Cranberry Bean, Great Northern Bean, Lima Bean, Navy Bean, and Pinto Bean.

Bacillus subtilis Solid Substrate Delivery System containing 3-75% of total biomass material on a dry weight basis: contains one or a combination of herbs, spices, seeds, Berries including Ashwagandha Root, Turmeric Rhizome, Milk Thistle Seed, Holy Basil Leaf, Ginger Rhizome, Oregano Leaf, Cinnamon Bark, Schizandra Berry, Amy Berry, Lemon Peel, and Green Tea,

Liquid Substrate Nutrient Stimulant: may contain for example 500-1,000 ml total liquid material consisting of a single or combination of juices from fruits, vegetables and herbs containing low or high levels of naturally occurring sugars such as Aloe Vera (Inner Leaf), Purple Carrot, Grape, Apple, Pomegranate, Beet, Parsley and Cabbage.

Lactobacillus plantarum Solid Substrate Delivery System containing 25-97% of total biomass material on a dry weight basis: may contain for example one or more of legumes, seeds, herbs and spices such as, Kidney Bean, Pumpkin Seed, Fava Bean, Chia Seed, Green Split Pea, Fax Seed, Green Lentil, Garbanzo Bean, Hemp Seed, Black Bean, Mung Bean, Adzuki Bean, Sesame Seed, Cranberry Bean, Great Northern Bean, Lima Bean, Navy Bean and Pinto Bean.

With the invention, as exemplified above, a “MicroBiome” results in which the bacterial biomass creates enhanced constituents, including not only probiotics generally but specific desirable enzymes, vitamins, minerals, essential fatty acids, polyphenolic antioxidants and organic adds. These enhanced constituents are objectively different and more biologically active than fermentation products created with standard growth media and substrata of the prior art.

Although the invention has been described above in connection with specific materials and methods, with reference to particular examples, the invention is only to be limited insofar as is set forth in the accompanying claims.

We claim:

1. A method for enhancing the probiotic constituency of edible or fermented foods, comprising growing at least one bacterium selected from the group consisting of Bacillus subtilis, Bacillus subtilis ssp Natto, Bacillus coagulans, and Lactobacillus plantarum, on a growth medium containing one or more of growth media selected from the group consisting of Kidney Bean; Pumpkin Seed; Fava Bean; Chia Seed; Green Split Pea; Flax Seed; Green Lentil; Garbanzo Bean; Hemp Seed; Black Bean; Mung Bean; Adzuki Bean; Sesame Seed; Cranberry Bean; Great Northern Bean; Lima Bean; Navy Bean; Pinto Bean; Black rice; Ashwaghandha; Cacao nibs; Purple Millet, Black Quinoa, Red Quinoa, Red Canihua, Cannabis Saliva/Indica flowers or leaves; Spirulina; Lemon Peel; Chili Pepper; Kelp; Alfalfa leaf; Watercress; Cilantro; Sage; Thyme; Parsley; Broccoli Seed; Mate; green Coffee Beans; Roasted Coffee Beans; Green Tea; Rhodiola; Siberian Ginseng; Sea Buckthorn Berry; Black Soy Bean; Olive leaf; Cabbage; Milk Thistle seed; Milk Thistle leaf; Turmeric; Bupleurum; Artichoke leaf; Dandelion leaf; Dandelion root; Hibiscus flower; Ginkgo leaf; Bacopa; Periwinkle; Peppermint; Hawthorn Berry; Cardamon; Garlic; Mung Bean; Astragalus Root; Echinacea purpurea root; Echinacea augustifolia root; Echinacea augustifolia leaf; Eider berries; Elder Flower; Goldenseal Leaf; Goldenseal root; Olive Leaf; Ginger; Orange Peel and Blossom; Clove; Cinnamon; Fennel; Kelp; Bitter Orange/Bergamot; Hibiscus Flower; Frankincense tears; Holy Basil; Celery seed; Bilberry leaf; Gymnema sylvestre leaf; Fennugreek; Bitter Melon; Saw Palmetto; Nettle; Red Clover Blossom; Black Cohosh; Chaste Tree Berries; Dong Quai Root; Hop Flowers; Licorice root; Wild Yam; Thyme Leaf; Oregano; Hyssop; Marjoram; American Ginseng; Panax Ginseng—red and white; Cruciferous vegetables; Schizandra; Goji Berry; Tribulus; Velvet Bean; Cranberry; Almonds; and Epimidium.

2. The growth method of claim 1, wherein said medium is supplemented by the addition of one or more of: Organic Carrot Juice and Concentrate; Organic Concord Grape Juice; Organic Pomegranate Juice and Concentrate; Organic Ginger Juice; Organic Blueberry Juice and/or Puree; Organic Acai Puree; Organic Plum Puree; Organic Raspberry Puree; Organic Tomato Juice and/or Puree; Organic Cranberry Juice; Organic Coconut Cream; Organic Coconut Water; Organic Cherry Juice; Organic Green Tea; Organic Apple Juice; Organic Vegetable Juice; Organic Pineapple Juice; Organic Aloe Vera Juice.

3. An edible composition in unit dosage form comprising Kidney Bean, Garbanzo Bean, Black Bean, Sunflower Seed, Turmeric, Green Tea, and Astralagas in a base of the juice from Aloe Vera Inner Leaf, fermented with Bactilis Subtilis and encapsulated into unit doses suitable for animals or humans.

4. An edible composition in unit dosage form comprising Kidney Bean, Pumpkin Seed, Fava Bean, Chia Seed, Green Split Pea, Flax Seed, Green Lentil, Garbanzo Bean, Hemp Seed, Black Bean, Mung Bean, Adzuki Bean, Sesame Seed, Cranberry Bean, Great Northern Bean, Lima Bean, Navy Bean, Pinto Bean, Turmeric, Green Tea and Chili Pepper in a base of combined organic Grape, Apple, Blueberry, Pomegranate, Blackberry, Cranberry, Lemon, Acerola Cherry, Celery, Purple Carrot, Green Leaf Lettuce, Red Beet, Purple Cabbage, Kale, Collards, Spinach, Watercress, Parsley juices with added Rose Hips, all of which have been inoculated and fermented with Bacillus subtilis prior to encapsulated into individual dosage forms suitable for animal or human consumption.
